(19) United States
(12) Patent Application Publication
(10) Pub. No.: US 2026/0137604 A1
(43) Pub. Date: May 21, 2026

(54) COSMETIC COMPOSITIONS

(71) Applicant: L'OREAL, Paris (FR)

(72) Inventors: Yon Jae YOON, Roselle, NJ (US); Jonathan FAIG, Sayreville, NJ (US)

(73) Assignee: L'OREAL, Paris (FR)

(21) Appl. No.: 18/952,148

(22) Filed: Nov. 19, 2024

(51) Int. Cl. A61K 8/06; A61Q 19/00

(52) U.S. Cl. CPC A61K 8/062; A61Q 19/008; A61K 2800/30; A61K 2800/436; A61K 2800/48; A61K 2800/594; A61K 2800/874

(57) ABSTRACT

Cosmetic compositions include one or more pearlescent pigments; and a thickening system including at least one synthetic polymer and at least one thickening polysaccharide; wherein the composition is free of emulsifiers; wherein the one or more pearlescent pigments and the thickening system form a stable suspension. The cosmetic compositions are in the form of a low viscosity, emulsifier-free emulsion (with pearlescent pigments in suspension while maintaining unique rheology compatible with rollerball cartridges.

### FIELD OF THE DISCLOSURE

The instant disclosure relates to stable cosmetic compositions in the form of a low viscosity, emulsifier-free emulsion (oil in water), containing pearlescent pigment in suspension while maintaining unique rheology compatible with rollerball cartridges. The instant disclosure also describes methods for treating skin with the cosmetic compositions.

### BACKGROUND

There are a variety of skin conditions that benefit from stable cosmetic compositions that may be applied and left on the skin to brighten skin, to improve the skin tone, long term firming among other possible benefits. Many skin care products provide active ingredients that address one or more of these or other skin issues, yet there are challenges in providing products that have sufficiently high amounts of actives to confer the desired benefits. Products that include high amounts of actives are often formulated with a high content of oils, emulsifiers or thickeners to stabilize the actives. Such formulations can be heavy and have a high viscosity.

### SUMMARY

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

The disclosed compositions described here presented a unique challenge. The challenge was to create stable cosmetic compositions that contained pearlescent pigments that could be suspended and that could be used with rollerball cartridges devices. Therefore, the compositions needed to be fluid enough to flow through the rollerball mechanism (hence, low viscosity) but also maintain a gel-like structure to prevent leakage. The inventors discovered that by combining pearlescent pigments and a specific thickening system containing at least one synthetic polymer and at least one thickening polysaccharide surprisingly presented a low viscosity and could be used with rollerball cartridges devices.

In various embodiments, a cosmetic composition is provided for use according to the instant disclosure that includes:
one or more pearlescent pigments; and
a thickening system including at least one synthetic polymer and at least one thickening polysaccharide;
wherein the composition is free of emulsifiers;
wherein the one or more pearlescent pigments and the thickening system form a stable suspension; and
wherein the thickening system is present in an amount sufficient to cause the cosmetic composition to be retained within a rollerball cartridge assembly without substantial leakage.

In certain embodiments, the cosmetic compositions may optionally include one or more emulsifiers, but preferably the cosmetic compositions are free or essentially free from emulsifiers.

Nonlimiting examples of pearlescent pigments include titanium mica coated with an iron oxide, mica coated with bismuth oxychloride, titanium mica coated with chromium oxide, nacreous pigments based on bismuth oxychloride, mica coated with titanium dioxide and iron oxides, mica coated with titanium dioxide, and mica coated with titanium dioxide and tin oxide, and combinations thereof. In various embodiments, the one or more pearlescent pigments are present from about 0.1 wt. % to about 5 wt. % based on the total weight of the composition. In one embodiment, the one or more pearlescent pigments is present from about 0.15 to about 4.8 wt. % based on the total weight of the composition.

In some embodiments, the cosmetic compositions may include a thickening system including at least one synthetic polymer and at least thickening polysaccharide.

Nonlimiting examples of synthetic polymer may include acrylates/C10-30 alkyl acrylate crosspolymer, hydroxyethyl acrylate/sodium acryloyldimethyl taurate copolymer (may be 89% active), polyacrylate crosspolymer-6, acrylamide/sodium acryloyldimethyltaurate copolymer, ammonium acryloyldimethyltaurate/steareth-25 methacrylate crosspolymer, acrylates/beheneth-25 methacrylate copolymer, ammonium polyacryloyldimethyl taurate, sodium polyacrylate, carbomer and the like, and combinations thereof. In some embodiments, the at least one synthetic polymer is present from about 0.10 to about 1.0 wt. % based on the total weight of the composition. In more embodiments, the at least one synthetic polymer is present from about 0.15 to about 0.9 wt. % based on the total weight of the composition.

Nonlimiting examples of thickening polysaccharide may include sclerotium gum, xanthan gum, cellulose gum, locust bean gum, carrageenan, modified celluloses (ethyl, isopropyl, methyl), acacia gum, sodium hyaluronate, and combinations thereof. In some embodiments, the at least one thickening polysaccharide is present from about 0.1 to about 0.4 wt. % based on the total weight of the composition.

In an aspect, the instant disclosure is directed to, among other things, a stable suspension. The stability of the suspension is determined by centrifugation at 900 rpm, at 25° C., for one hour. In some embodiments, the stable suspension does not show significant separation of the components in the process.

In some embodiments, the composition comprises a viscosity of from about 20 Deviation Unit (UD) to about 80 Deviation Unit (UD), measured at 25° C. using a Rheomat R180 viscometer equipped with a M2 spindle at a shear rate of 200 rpm.

In some embodiments, the composition comprises a viscosity ranging from about 0.15 Pa·s to about 0.35 Pa·s, measured at 25° C., at a shear rate of 200 1/s.

In various embodiments, the thickening system amount comprises from about 0.10 to about 1.0 wt. % based on the total weight of the composition of at least one synthetic polymer; and from about 0.1 to about 0.4 wt. % based on the total weight of the composition of at least one thickening polysaccharide; the said amount is sufficient to achieve a viscosity of about 0.15 Pa·s to about 0.34 Pa·s, measured at 25° C. at a shear rate of 200 1/s. The viscosity of about 0.15 Pa·s to about 0.34 Pa·s, measured at 25° C. at a shear rate of 200 1/s is sufficient to cause the composition to be retained within a rollerball cartridge assembly without substantial leakage.

In some embodiments, the viscosity of about 20 Deviation Unit (UD) to about 80 Deviation Unit (UD), measured at 25° C. using a Rheomat R180 viscometer equipped with a M2 spindle at a shear rate of 200 rpm is sufficient to cause the composition to be retained within a rollerball cartridge assembly without substantial leakage.

In various embodiments, the cosmetic composition is retained within a rollerball cartridge assembly without substantial leakage when the rollerball cartridge is in an inverted, idle position for one month at 45° C.

In some embodiments, the cosmetic composition comprises a gel composed of a gelling agent of the thickening system, a solvent, and the one or more pearlescent pigments.

Nonlimiting examples of solvent may include at least one of the solvent chosen from glycerin, C2-C6 mono-alcohols, polyols (polyhydric alcohols), glycols, and a mixture thereof. In some embodiments, the solvent is present from about 5 to about 30 wt. % based on a total weight of the cosmetic composition.

In various embodiments, the compositions may optionally include one or more skin active agents.

In some embodiments, the one or more skin active agents are present at a level higher than 10 wt. % based on a total weight of the composition.

In various embodiments, the cosmetic composition may have a viscosity of about 0.15 to about 0.35 Pa·s at 25° C., and shear rate of 200 1/s at 25° C.

In an embodiment, the cosmetic compositions form part of a device comprising a cosmetic composition according to the instant disclosure. In an embodiment, the cosmetic compositions are received in a kit, which is a device, for example, a device that dispenses the cosmetic compositions. In an embodiment, the device with rollerball cartridges comprises cosmetic composition that is compatible with the said rollerball cartridges, the cosmetic composition comprising:
a viscosity below 0.34 Pa·s at 25° C., and shear rate of 200 1/s at 25° C.; and
wherein the cosmetic composition comprises:
one or more pearlescent pigments; and
a thickening system including at least one synthetic polymer and at least one thickening polysaccharide;
wherein the composition is free of emulsifiers;
wherein the one or more pearlescent pigments and the thickening system form a stable suspension; and
wherein the thickening system is present in an amount sufficient to cause the composition to be retained within a rollerball cartridge assembly without substantial leakage.

Another aspect of the instant disclosure relates to methods for treating skin. The methods include applying the cosmetic composition according to the instant disclosure to the skin.

The disclosed compositions described here presented a unique challenge. The challenge was to create stable cosmetic compositions that contained pearlescent pigments that could be suspended and that could be used with rollerball cartridges devices. Therefore, the compositions needed to be fluid enough to flow through the rollerball mechanism (hence, low viscosity) but also maintain a gel-like structure to prevent leakage. The inventors discovered that by combining pearlescent pigments and a thickening system containing at least one synthetic polymers and at least one thickening polysaccharide surprisingly presented a low viscosity and could be used with rollerball cartridges devices. The thickening system being present in an amount sufficient to cause the composition to be retained within a rollerball cartridge assembly without substantial leakage.

Other features and iterations of the invention are described in more detail.

### DETAILED DESCRIPTION

The instant compositions are directed to be used with a system, a device and methods with physical properties suitable for being dispense using roller ball cartridges. Some of the challenges were that the cosmetic compositions would not leak from the roller ball cartridges, that the cosmetic compositions would not separate and that the cosmetic compositions would not form a blockage inside the roller ball cartridges which would affect the performance.

In one embodiment suitable to the cosmetic compositions of the instant disclosure comprise:
one or more pearlescent pigments; and
a thickening system including at least one synthetic polymer and at least one thickening polysaccharide;
wherein the composition is free of emulsifiers;
wherein the one or more pearlescent pigments and the thickening system form a stable suspension; and
wherein the thickening system is present in an amount sufficient to cause the cosmetic composition to be retained within a rollerball cartridge assembly without substantial leakage.

### Pearlescent Pigments

In some embodiments, the composition of the instant disclosure comprises at least one or more pearlescent pigments.

In various embodiments, the composition may include one or more pearlescent pigments selected from the group consisting of titanium mica coated with an iron oxide, mica coated with bismuth oxychloride, titanium mica coated with chromium oxide, nacreous pigments based on bismuth oxychloride, mica coated with titanium dioxide and iron oxides, mica coated with titanium dioxide, and mica coated with titanium dioxide and tin oxide, and combinations thereof. In some embodiments, the one or more pearlescent pigments according to the instant disclosure may be present from about 0.1 wt. % to about 5 wt. % based on the total weight of the composition. In some embodiments, the one or more pearlescent pigments according to the instant disclosure may be present from about 0.15 to about 4.8 wt. % based on the total weight of the composition

In some embodiments, “pearlescent pigments”, which may also be called “nacres”, may be colored particles of any form, which may or may not be iridescent, especially produced by certain molluscs in their shell, or alternatively synthesized, and which have a color effect via optical interference.

Preferably, the pearlescent pigment has a volume average size (D50) in the range of 1-20 μm, preferably in the range of 5-15 μm.

Preferably, the pearlescent pigment has a volume average size (D90) in the range of 1-25 μm, preferably in the range of 5-20 μm.

The volume average size (D90) is a parameter for particle size distribution, referring to the maximum particle diameter below which 90% of the sample volume exists.

The pearlescent pigment with a limited particle size and a colored reflection can contribute to color correction of a keratin substance such as skin by mixing the colored reflection with the original color of the keratin substance, and to natural appearance.

The pearlescent pigment can be in any shape. For example, it is possible to use a pearlescent pigment in the form of a plate with an aspect ratio of at least 5, preferably more than 10, more preferably more than 20, and even more preferably more than 50. The aspect ratio can be determined by the average thickness and the average length according to the formula: aspect ratio=length/thickness.

If a plate-like particle is used for the pearlescent pigment, it is preferable that the plate-like particle have a length of 15 μm or less, preferably 12 μm or less, and more preferably 10 μm or less.

Examples of the pearlescent pigment that may be mentioned include titanium mica coated with an iron oxide, mica coated with bismuth oxychloride, titanium mica coated with chromium oxide, nacreous pigments based on bismuth oxychloride, mica coated with titanium dioxide and iron oxides, mica coated with titanium dioxide, and mica coated with titanium dioxide and tin oxide. They may also be mica particles at the surface of which are superposed at least two successive layers of metal oxides and/or of organic dyestuffs.

These particles may be chosen from particles containing a natural or synthetic substrate at least partially coated with at least one layer of at least one metal oxide, chosen, for example, from titanium oxides, especially TiO2, iron oxides, especially Fe2O3, tin oxides, chromium oxides, barium sulfate and the following materials: MgF2, CrF3, ZnS, ZnSe, SiO2, Al2O3, MgO, Y2O3, SeO3, SiO, HfO2, ZrO2, CeO2, Nb2O5, Ta2O5, MoS2, and mixtures or alloys thereof.

The pearlescent pigment may have a reflection with a color such as blue, yellow, pink, red, bronze, orange, brown, gold and copper.

It may be preferable that the pearlescent pigment comprises mica and metal oxide, preferably titanium oxide, iron oxides, and/or tin oxide.

Nonlimiting examples of pearlescent pigment that may be mentioned in particular, alone or as a mixture, include pearlescent pigment including the following elements:
mica-titanium oxide (for example sold under the commercial reference Timica Sparkle 110P® from BASF, Flamenco Blue® from BASF, Candurin Brown Amber® from Merck, Pearlescent Pigment Prestige Gold® from Sudarshan);
mica-titanium oxide coated with N-lauroyl-L-lysine (for example sold under the commercial reference LLD-10 Flamenco Violet® from Daito Kasei Kogyo);
mica-iron oxide (for example sold under the commercial reference Pearlescent Pigment Prestige Soft Bronze® from Sudarshan, Colorona Bronze Sparkle® from Merck, Timica Golden Bronze® from BASF, Candurin Light Gold® from Merck, Duocrome YR 422 C® from BASF, Gemtone Tan Opal G 005® from BASF);
mica-titanium oxide-iron oxide (for example sold under the commercial reference Timica Brilliant Gold 212 G Gemtone Goldstone G0014® or Timica Golden Bronze® from BASF);
mica-bismuth oxychloride-iron oxide (for example sold under the commercial reference Chroma-Lite Yellow® from BASF);
mica-titanium oxide-chromium oxide-iron oxide (for example sold under the commercial reference Gemtone Jade® from BASF);
mica-titanium oxide-tin oxide (for example sold under the commercial reference Flamenco Super Blue 630 Z® from BASF, Helios R100R® from Topy);
mica-silica-titanium oxide (for example sold under the commercial reference Timiron Splendid Red® from Merck);
platelets of sodium calcium borosilicate coated with titanium oxide and tin oxide (for example sold under the commercial reference Reflecks Rays of Red G430L® from BASF);
platelets of sodium calcium borosilicate coated with tin oxide and iron oxide (for example sold under the commercial reference Mirage Sparkling Champagne® from Eckart);
platelets of calcium aluminium borosilicate coated with titanium oxide and tin oxide, optionally treated with silica (for example sold under the commercial reference Ronastar® from Merck);
platelets of calcium aluminium borosilicate coated with silica oxide and tin oxide (for example sold under the commercial reference Ronastar Golden Jewel SQ® from Merck);
glass powder coated with titanium oxide (for example sold under the commercial reference Reflecks Dimensions Glittering Gold G230S® from BASF or Metashine MC1120RY® from NSG);
alumina and titanium oxide (for example sold under the commercial reference Spectraflex Focus Red C88-1031® from Sun);
bronze powder (for example sold under the commercial reference Visionaire Bright Sunflower Gold® from Eckart);
synthetic fluorophlogopite and magnesium silicate and titanium dioxide and tin oxide (for example sold under the commercial reference Sunshine Daybreak Blue® from Sun Chemical).

For the purposes of the instant disclosure, the term “mica” includes natural micas and synthetic micas.

In some embodiments, the composition of the instant disclosure comprises up to 5 wt. % of at least one or more pearlescent pigments, based on the total weight of the composition. In one or more embodiments, the composition of the instant disclosure comprises from about 0.1 to about 5 wt. % based on the total weight of the composition. In one embodiment, the composition of the instant disclosure comprises 0.15 to about 4.8 wt. % based on the total weight of the composition.

In some embodiments, any one of the least one or more pearlescent pigments is present in a range from about 0.1% to about 5.0%, by weight, based on the total weight of the cosmetic composition. In some embodiments, the total amount of the at least one or more pearlescent pigments is not more than about 5.0%, total, by weight, based on the total weight of the cosmetic composition. In some embodiments, the total amount of the least one or more pearlescent pigments is at least about 0.1%, or about 0.15%, or about 0.2%, or about 0.3%, or about 0.4%, or about 0.5%, or about 0.6%, or about 0.7% or about 0.8%, or about 1%, or about 1.1%, or about 1.2%, or about 1.3%, or about 1.4%, or about 1.5%, or about, 1.6%, or about 1.7%, or about 1.8%, or about 1.9%, or about 2.0%, or about 2.1%, or about 2.2%, or about 2.3%, or about 2.4%, or about 2.5%, or about 2.6%, or about 2.7%, or about 2.8%, or about 2.9%, or about 3.0%, or about 3.1%, or about 3.2%, or about 3.3%, or about 3.4%, or about 3.5%, or about 3.6%, or about 3.7%, or about 3.8%, or about 3.9%, or about 4.0%, or about 4.1%, or about 4.2%, or about 4.3%, or about 4.4%, or about 4.5%, or about 4.6%, or about 4.7%, or about 4.8%, or about 4.9%, or about 5.0% or any suitable combination, sub-combination, range, or sub-range thereof by weight, based on the weight of the fluid emulsion cosmetic composition. One of ordinary skill in the art, however, will appreciate that other ranges are within the scope of the invention.

The red pearlescent pigment can show a red reflection on a black background. Here, the term “red” means visible light with a wavelength of from about 640 nm to about 780 nm.

As illustrations of red pearlescent pigment that may be introduced into the composition according to the present invention, mention may be made of TITANIUM DIOXIDE (and) MICA sold in the name of RONASTAR MAGIC RED by the company MERCK.

### Thickening System

In accordance with the disclosure, the composition includes a thickening system constituted by a combination of polymeric thickeners that include at least one synthetic polymer, and at least one thickening polysaccharide.

In some embodiments, the thickening system comprises at least one synthetic polymer selected from the group consisting of acrylates/C10-30 alkyl acrylate crosspolymer, hydroxyethyl acrylate/sodium acryloyldimethyl taurate copolymer (may be 89% active), polyacrylate crosspolymer-6, acrylamide/sodium acryloyldimethyltaurate copolymer, ammonium acryloyldimethyltaurate/steareth-25 methacrylate crosspolymer, acrylates/beheneth-25 methacrylate copolymer, ammonium polyacryloyldimethyl taurate, sodium polyacrylate, carbomer, and combinations thereof. In some embodiments, the at least one synthetic polymer is present from about 0.10 to about 9.5 wt. % based on the total weight of the composition. In other embodiments, the at least one synthetic polymer is present from about 0.2 to about 9.0 wt. % based on the total weight of the composition.

In some embodiments, the total amount of the least one synthetic polymer is at least about 0.10%, or about 0.2%, or about 0.4% or about 0.6% or about 0.8%, or about 1.0% or about 1.5% or about 2.0% or about 2.5% or about 3.0% or about 3.5%, or about 4.0%, or about 4.5%, or about 5.0%, or about 5.5%, or about 6.0%, or about 6.5%, or about 7.0%, or about 7.5%, or about 8.0%, or about 8.5%, or about 9.0%, or about 9.5%, or any suitable combination, sub-combination, range, or sub-range thereof by weight, based on the weight of the fluid emulsion cosmetic composition. One of ordinary skill in the art, however, will appreciate that other ranges are within the scope of the invention.

In some embodiments, the thickening system comprises the at least one thickening polysaccharide selected from the group consisting of gums, cellulose derivatives, and combinations thereof. In some embodiments, the thickening system comprises the at least one thickening polysaccharide selected from the group consisting of sclerotium gum, xanthan gum, cellulose gum, locust bean gum, carrageenan, modified celluloses (ethyl, isopropyl, methyl), acacia gum, sodium hyaluronate, and combinations thereof. In one embodiment, the at least one thickening polysaccharide is present from about 0.1 to about 0.4 wt. % based on the total weight of the composition.

In some embodiments, any one of the thickening polysaccharides is present in a range from about 0.2% to about 0.35%, by weight, based on the total weight of the composition. In some embodiments, the total amount of the thickening polysaccharide present in the cosmetic composition is in an amount from about from about 0.15% to about 0.4%, or from about 0.2% to about 0.4%, or from about 0.2% to about 0.3%, or from about 0.25% to about 0.35%, or about 0.39%, or any suitable combination, sub-combination, range, or sub-range thereof by weight, based on the weight of the cosmetic composition. One of ordinary skill in the art, however, will appreciate that other ranges are within the scope of the invention.

In one embodiment, thickeners includes any organic or inorganic compound that is capable of increasing the viscosity of the composition according to the instant disclosure.

The thickeners in accordance with the instant disclosure are preferably water-soluble/swellable.

In one embodiments, a water soluble thickener may include any thickener that can be fully dissolved in molecular form or that is miscible in a liquid aqueous phase or that can be dissolved in colloidal form (for example in micellar form) in a liquid aqueous phase.

As thickeners that may be used in the compositions according to the instant disclosure, mention may be made of:
polyacrylic acids of the type such as Synthalen K®; polyacrylic acids crosslinked with a pentaerythrityl ally ether, a sucrose allyl ether or a propylene allyl ether of Carbomer type; copolymers of at least two monomers chosen from acrylic acid, methacrylic acid or an ester thereof of INCI name Acrylates copolymer, in particular the product sold under the name Carbopol Aqua SF1® by the company Lubrizol Advanced Materials, Inc., copolymers of acrylic acid and of acrylamide, sold in the form of their sodium salt under the names Reten® by the company Hercules; crosslinked polyacrylic acid/C10-C30 alkyl acrylate copolymers of INCI name: Acrylates/C10-C30 alkyl acrylate crosspolymer, sold under the trade name Pemulen® or Carbopol 1382® sold by the company Lubrizol Advanced Materials, Inc., an acrylamidomethylpropanesulfonic acid polymer partially neutralized with ammonia and crosslinked, of INCI name: Ammonium polyacryloyldimethyl taurate sold under the trade name Hostacerin AMPS® by the company Clariant, AMPS®/acrylamide copolymers of Sepigel® or Simulgel® type sold by the company SEPPIC, and AMPS®/polyoxyethylenated alkyl methacrylate copolymers (crosslinked or non-crosslinked), and mixtures thereof.

Other examples of thickening polymers that may be mentioned include: proteins, for instance proteins of plant origin such as wheat or soybean proteins; proteins of animal origin such as keratins, for example keratin hydrolysates and sulfonic keratins; anionic, cationic, amphoteric or nonionic chitin or chitosan polymers; cellulose polymers such as hydroxyethylcellulose, hydroxypropylcellulose, methylcellulose, ethyl-hydroxyethylcellulose and carboxymethylcellulose, and also quaternized cellulose derivatives; vinyl polymers, for instance polyvinylpyrrolidones, copolymers of methyl vinyl ether and of malic anhydride, the copolymer of vinyl acetate and of eratonic acid, copolymers of vinylpyrrolidone and of vinyl acetate; copolymers of vinylpyrrolidone and of caprolactam; polyvinyl alcohol; associative polyurethanes such as the C16-OE120-C166® polymer from the company Servo Delden (sold under the name SER AD FXII00®, this molecule bearing a urethane function and having a weight-average molecular weight of 1300), OE being an oxyethylene unit, Rheolate 205® bearing a urea function, sold by the company Rheox, or Rheolate 208® or 204® (these polymers being sold in pure form) or DW 1206B® from Rohm & Haas bearing a C20 alkyl chain and a urethane bond, sold at 20% solids in water. It is also possible to use solutions or dispersions of these associative polyurethanes, especially in water or in aqueous-alcoholic medium. Examples of such polymers that may be mentioned include SER AD FXI 010, SER AD FX1035® and SER AD FX1070® from the company Servo Delden, and Rheolate 255®, Rheolate 278® and Rheolate 244® sold by the company Rheox. It is also possible to use the products DW 1206F® and DW 12061®, and also Acrysol RM 184® or Acrysol 44® from the company Rohm & Haas, or alternatively Borchigel LW 44® from the company Borchers; optionally modified polymers of natural origin, such as: gum arabic, guar gum, xanthan derivatives, karaya gum; alginates and carrageenans; glycosaminoglycans, hyaluronic acid and derivatives thereof; shellac resin, sandarac gum, dammar resins, elemi gums and copal resins; deoxyribonucleic acid; mucopolysaccharides such as hyaluronic acid and chondroitin sulfates, and mixtures thereof.

Use will more particularly be made of a thickener chosen from: copolymers of at least two monomers chosen from acrylic acid, methacrylic acid or an ester thereof of INCI name Acrylates copolymer, in particular the product sold under the name Carbopol Aqua SF1® by the company Lubrizol Advanced Materials, Inc., polyacrylic acid/C10-C30 alkyl acrylate crosslinked copolymers of INCI name: Acrylates/C10-C30 alkyl acrylate crosspolymer, such as the products sold under the trade names Pemulen TRI and Pemulen TR2® or Carbopol 1382®, Carbopol ETD 2020 Polymer®, Carbopol 1342 Polymer®, Carbopol SC 200®, Carbopol SC 500 Polymer®, Carbopol Ultrez 20 Polymer®, Carbopol Ultrez 21 Polymer® from the company Lubrizol Advanced Materials, Inc.; an acrylamidomethylpropanesulfonic acid polymer partially neutralized with ammonia and cross inked, of INCI name: Ammonium polyacryloyldim-ethyl taurate sold under the trade name Hostacerin AMPS® by the company Clariant, and mixtures thereof.

The thickener(s) may preferably be used in a proportion of from about 0.1% to about 1.1% by weight weight relative to the total weight of the composition, especially from about 0.1% to about 1.09% by weight relative to the total weight of the composition.

In some embodiments, the thickening system amount comprises from about 0.10 to about 1.0 wt. % based on the total weight of the composition of at least one synthetic polymer; and from about 0.1 to about 0.4 wt. % based on the total weight of the composition of at least one thickening polysaccharide; the said amount is sufficient to achieve a viscosity of about 20 UD to 80 UD, measured at 25° C. using a Rheomat R180 viscometer equipped with a M2 spindle at a shear rate of 200 rpm.

In some embodiments, the thickening system amount comprises from about 0.10 to about 1.0 wt. % based on the total weight of the composition of at least one synthetic polymer; and from about 0.1 to about 0.4 wt. % based on the total weight of the composition of at least one thickening polysaccharide; the said amount is sufficient to achieve a viscosity of about 0.15 to about 0.35 Pa·S at 25° C., and shear rate of 200 s-1 at 25° C.

### Emulsifier Free

Non-limiting examples of “surfactants” and “emulsifiers” include salts of the surfactants and emulsifiers. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

In an embodiment “substantially free” or “essentially free” denotes that the specific material may be present in small amounts that do not materially affect the basic and novel characteristics of the claimed invention. For instance, there may be less than 2% by weight of a specific material added to a composition, based on the total weight of the composition (provided that an amount of less than 2% by weight does not materially affect the basic and novel characteristics of the claimed invention). Similarly, a composition “substantially free” or “essentially free” of a stated material may include less than 1.5 wt. %, less than 1 wt. %, less than 0.5 wt. %, less than 0.1 wt. %, less than 0.05 wt. %, or less than 0.01 wt. %, or none of the specified material. The term “substantially free” or “essentially free” as used herein may also mean that the specific material is not added to the composition but may still be present in a raw material that is included in the composition.

Furthermore, all components that are positively set forth in the instant disclosure may be negatively excluded from the claims, e.g., a claimed composition may be “free,” “essentially free” (or “substantially free”) of one or more components that are positively set forth in the instant disclosure.

### Stable Suspension

In some embodiments, the cosmetic composition according to the instant disclosure is a stable suspension formed by the associations of pearlescent pigments and a thickening system including at least one synthetic polymer and at least one thickening polysaccharide; and wherein the composition is free of emulsifiers. With respect to physical stability, in certain embodiments, the compositions are homogenous, and the pearlescent pigments do not show substantial settlement or phase separation.

In some embodiments, stable suspensions are defined as being stable after the compositions are centrifuged at 900 rpm, at 25 C for one hour. In various embodiments, the stable suspension does not show significant separation of the pearlescent pigments and a thickening system.

In some embodiments, the thickening system is present in an amount sufficient to cause the composition to be retained within a rollerball cartridge assembly without substantial leakage. Nonlimiting examples of rollerball cartridge assembly without substantial leakage may be when the roller ball cartridge is in an inverted, idle position for one month at 45 C.

### Viscosity

For proper performances within devices with fluiditic pathways and dispensing without leaking, it requires the optimization of the properties of the material including the viscosity.

The viscosity of the compositions according to the instant disclosure can be measured and have a range from about 0.15 Pa·s to about 0.35 Pa·s at 25° C., and shear rate of 200 1/s at 25° C.

In some embodiments, the viscosity is in a range from about 0.15 Pa·s, 0.16 Pa·s, 0.17 Pa·s, 0.18 Pa·s, 0.19 Pa·s, 0.20 Pa·s, 0.21 Pa·s, 0.22 Pa·s, 0.23 Pa·s, 0.24 Pa·s, 0.25 Pa·s, 0.26 Pa·s, 0.27 Pa·s, 0.28 Pa·s, 0.29 Pa·s, 0.30 Pa·s, 0.31 Pa·s, 0.32 Pa·s, 0.33 Pa·s, 0.34 Pa·s, to about 0.35 Pa·s, or any suitable combination, sub-combination, range, or sub-range thereof. One of ordinary skill in the art, however, will appreciate that other ranges are within the scope of the invention.

The viscosity of the compositions according to the instant disclosure can be measured at 25° C. using a Rheomat R180 viscometer equipped with a M2 spindle at a shear rate of 200 rpm. In some embodiments, the compositions of the instant disclosure have a viscosity of from about 20 UD (Deviations Units) to about 80 UD, measured at 25° C. using a Rheomat R180 viscometer equipped with a M2 spindle at a shear rate of 200 rpm.

In some embodiments, the viscosity is in a range from about 20 UD, 21 UD, 22 UD, 23 UD, 24 UD, 25 UD, 26 UD, 27 UD, 28 UD, 29 UD, 30 UD, 31 UD, 32 UD, 33 UD, 34 UD, 35 UD, 36 UD, 37 UD, 38 US, 39 UD, 40 UD to about 40 UD, 41 UD, 42 UD, 43, UD, 44 UD, 45, UD, 46 UD, 47 UD, 48 UD, 49 UD, 50 UD, 51 UD, 52, UD 53 UD, 54 UD, 55 UD, 56, UD, 57, UD, 58 UD, 59 UD, 60 UD, 61 UD, 62, UD, 63, UD, 64 UD, 65, UD, 66 UD, 67 UD, 68 UD, 69 UD, 70 UD, 71 UD, 72 UD, 73 UD, 74 UD, 75 UD, 76 UD, 77 UD, 78 UD, 79 UD, to about 80 UD, or any suitable combination, sub-combination, range, or sub-range thereof. One of ordinary skill in the art, however, will appreciate that other ranges are within the scope of the invention.

### Solvent

In some embodiments, the compositions according to the instant disclosure comprise a gelling amount of the thickening system, a solvent and the one or more pearlescent pigments.

Nonlimiting examples of solvents include glycerin, C2-C6 mono-alcohols, polyols, glycols, glycol ethers (for example, monomethyl, monoethyl, and monobutyl ethers of ethylene glycol or propylene glycol), propylene glycol, butylene glycol, hexylene glycol, caprylyl glycol, dipropylene glycol, alkyl ethers of diethylene glycol such as monoethyl ether or monobutyl ether of diethylene glycol (e.g., diethylene glycol monobutyl ether and diethylene glycol monoethyl ether), dipropylene glycol, and alkyl ethers of diethylene glycol, for example monoethyl ether or monobutyl ether of diethylene glycol.

In various embodiment, one or more solvents are selected from alkanediols (polyhydric alcohols or polyols) such as glycerin, 1,2,6-hexanetriol, trimethylolpropane, ethylene glycol, propylene glycol, diethylene glycol, triethylene glycol, caprylyl glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

Nonlimiting examples of polyols (polyhydric alcohols) include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a combination thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, of combinations thereof.

The total amount of the one or more solvents in the composition will vary but is typically about 5 to about 30 wt. %, based on a total weight of the composition. In further embodiments, the composition includes about 5%, or about 6%, or about 7%, or about 8%, or about 9%, or about 10%, or about 11%, or about 12% or about 13%, or about 14%, or about 15%, or about 16%, or about 17%, or about 18%, or about 19%, or about, 20%, or about 21%, or about 22%, or about 23%, or about 24%, or about 25%, or about 26%, or about 27%, or about 28%, or about 29%, or about 30%, or any suitable combination, sub-combination, range, or sub-range thereof by weight, based on the weight of the fluid emulsion cosmetic composition. One of ordinary skill in the art, however, will appreciate that other ranges are within the scope of the invention.

### Skin Active Agents

Skin active agents are ingredients included in the cosmetic composition to benefit, treat, or improve the health, appearance, or integrity of the skin. Skin active ingredients are often classified according to their function. Nonlimiting examples include anti acne compounds, anti-inflammatory compounds, skin renewal compounds, compounds that improve skin barrier function, depigmenting compounds, anti-wrinkle compounds, anti-aging compounds, and the like. For example, skin active agents include rosacea inhibitory agents (e.g., metronidazole, sulfacetamide, sodium sulfacetamide, sulfur, dapson, doxycycline, minocycline, clindamycin, clindamycin phosphate, erythromycin, tetracylclines, azelaic acid, calcium dobesilate, maleic acid, and any compatible combinations thereof); α-adrenergic receptor agonists (e.g., clonidine, amphetamine, doxtroamphetamine, apraclonidine, dipivefrin, α-methyldopa, oxymetazoline, oxymetazoline hydrochloride, methoxamine, metaraminol, medetomidine, dexmedetomidine, ethylnorepinephrine, guanfacine, guanabenz, phenylephrine, phenylephrine hydrochloride, ephedrine, epinine, epinephrine, ethylnorepinephrine, levarterenol, lofexidine, norepinephrine, norphenylephrine, norephedrine, phenylpropanolamine, pemoline, propylhexadrine, pseudoephedrine, methamphetamine, α-methylnorepinephrine, methylphenidate, mephentermine, midodrine, mivazerol, moxonidine, desglymidodrine, tetrahydrozoline, tetrahydrozoline hydrochloride, cirazoline, amidephrine, brimonidine, brimonidine tartrate, naphazoline, isoproterenol, xylazine, xylometazoline, and/or tizanidine); chemicals and botanical extracts with vasoconstrictor properties including, but not limited to, corticosteroids, ephedrine, pseudoephedrine, caffeine, and/or escin; Ephedra, Ephedra sinica, Hamamelis viginiana, Hydrastis canadensis, Lycopus virginicus, Aspidosperma quebracho, Cytisus scoparius, Raphanus sativus linn (radish leave extracts), horse chestnut extracts, dandelion extracts, etc.

In a preferred embodiment, the skin active agents are selected from ceramides, ceramide precursors, free fatty acids, linoleic/oleic acids, cholesterol, cholesterol sulfate, b-glucan, carob seed extract, eperua falcata extract, amino acids, niacinamide and its derivatives, hyaluronic acid & its derivatives, glycerin, allantoin, squalane, omega fatty acids, or combinations thereof.

Additional nonlimiting examples include chemicals or botanical extracts with anti-inflammatory properties (e.g., corticosteroids (for short term use)), non-steroidal anti-inflammatory drugs, linoleic acid, linolenic acid, bisabolol, glycyrrhetinic acid, glycerin, plant extracts with anti-inflammatory properties (i.e., tea extracts, chamomile extracts), anti-inflammatory interleukins (e.g., II-1 ra); isoprenylcystein analogues (i.e., N-acetyl-S-farnesyl-L-cysteine), aromatic aldehydes with anti-inflammatory properties (e.g., 4-ethoxy benzaldehyde), etc., as well as any compatible combinations thereof); chemicals or botanical extracts; chemicals or botanical extracts with anti-microbial properties (e.g., antibiotics including, but not limited to gentamicin, penicillins, cephalosporins, quinolones, ciprofloxacin, and/or novobiocin); chemicals or botanical extracts with anti-fungal properties (e.g., ketoconazole, naftifine hydrochloride, oxiconazole nitrate, sulconazole nitrate, urea, terbinafine hydrochloride, selenium sulfide, etc.); chemicals or botanical extracts with anti-dandruff properties; chemicals or botanical extracts with anti-seborrheic properties; keratolytic agents or botanical extracts with keratolytic properties (i.e., alpha-hydroxy acids; beta-hydroxy acids, poly-hydroxy acids, urea, salicylic acid, etc.); chemicals with astringent properties; serine protease inhibitors; saturated dicarboxylic acids; alpha hydroxy acids (e.g., glycolic acids, lactic acid, malic acid, citric acid, tartaric acid, etc.); beta hydroxy acids (e.g., carnitine, 3-hydroxybutyric acid, 3-hydroxypropionic acid, β-hydroxy β-methylbutyric acid, salicylic acid, etc.).

Additional nonlimiting examples of skin active agents include retinoic acid, tretinoin, isotretinoin, adapalene, retinol, and/or derivatives; benzoyl peroxide; dapsone; kinetin (N6-furfuryladenine) and derivatives (e.g., furfurylaminotetrahydro-pyranyladenine); niacinamide (nicotinamide), and combinations thereof. Even further nonlimiting examples of skin active agents include metronidazole, sulfacetamide, sodium sulfacetamide, sulfur, tetracylines, doxycycline, clindamycin, clindamycin phosphate, erythromycin, and/or minocycline, azelaic acid, calcium dobesilate, caffeine, theobromine, theophylline and/or a derivative thereof (i.e., xanthines), vitamin A, vitamin B1, vitamin B2, vitamin B3, vitamin B5, vitamin B6, vitamin B7, vitamin B9, vitamin B12, vitamin C, vitamin D, vitamin E and vitamin K, creatine, carnitine, and essential fatty acids such as linoleic acid and/or linolenic acid, zinc salts such as, for example, zinc sulfate, zinc chloride, zinc glycinate, zinc gluconate, zinc-histidine, zinc L-2-pyrrolidone-5-carboxylate (zinc PCA), zinc salt of linoleic acid, zinc salt of linolenic acid, zinc salt of azelaic acid, zinc peptides, zinc oxide, copper salts including, but not limited to, copper sulfate, copper chloride, copper glycinate, copper gluconate, copper-histidine, copper L-2-pyrrolidone-5-carboxylate (copper PCA), copper salt of linoleic acid, copper salt of linolenic acid, copper salt of azelaic acid, copper peptides,

In various embodiments, the one or more skin active agents are optionally selected from madecassoside, retinoic acid, benzoyl peroxide, sulfur, vitamin B6 (pyridoxine or) chloride, selenium, samphire, cinnamon extract blends, zinc gluconate, zinc pyrrolidonecarboxylate (or zinc pidolate), zinc lactate, zinc aspartate, zinc carboxylate, zinc salicylate, zinc cysteate, copper and copper pidolate as Cuivridone Solabia, extracts from plants of Arnica montana, Cinchona succirubra, Eugenia caryophyllata, Humulus lupulus, Hypericum perforatum, Mentha pipenta, Rosmarinus officinalis, Salvia officinalis and Thymus vulgaris, extracts of meadowsweet (Spiraea ulmaria), and mixtures thereof.

In various embodiments, one or more skin active agents may be selected from adenosine, 2-[4-(2-hydroxyethyl)piperazin-1-yl]ethanesulfonic acid (HEPES), hyaluronic acid, lanolin, citric acid, malic acid, lactic acid, tartaric acid, salicylic acid, vitamin C, a vitamin, a retinoid, retinal, retinoic acid, a carotenoid, an amino acid, a protein, an enzyme, and a coenzyme. In some cases the active ingredient is adenosine.

The skin active agent may be selected from lactic acid, hyaluronic acid, AHA, BHA, sodium pidolate, serine, sodium lactate, ectoin and its derivatives, chitosan and its derivatives, collagen, plankton, an extract of Imperata cylindra sold under the name Moist 24 by Sederma, homopolymers of acrylic acid as Lipidure-HM of NOF Corporation, beta-glucan and in particular sodium carboxymethyl beta-glucan Mibelle-AG-Biochemistry, a mixture of oils passionflower, apricot, corn, and rice bran sold by Nestle under the name NUTRALIPIDS, a C-glycoside derivatives, in particular the C-13-D-xylopyranoside-2-hydroxypropane, for example, as the product produced by the company Chimex under the trade name “MEXORYL SBB”, a rose hip oil marketed by Nestle, a micro-algae extract Prophyridium cruentum enriched with zinc, marketed under the name by VINCIENCE ALGUALANE ZINC spheres of collagen and chondroitin sulfate of marine origin (Atelocollagen) sold by the company Engelhard Lyon under the name MARINE FILLING SPHERES, hyaluronic acid spheres such as those marketed by Engelhard Lyon, and arginine.

Depigmenting agents include vitamin C and its derivatives and especially vitamin CG, CP and 3-O ethyl vitamin C, alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, ceramides and their counterparts, derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include vitamin C and its derivatives and especially vitamin CG, CP and 3-O ethyl vitamin C, alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

Useful skin active agents include adenosine and its derivatives and retinol and its derivatives such as retinol palmitate, ascorbic acid and its derivatives such as magnesium ascorbyl phosphate and ascorbyl glucoside; tocopherol and derivatives thereof such as tocopheryl acetate, nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the a-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof.

As adenosine derivatives include especially non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside.

Other derivatives include adenosine receptor agonists such as adenosine adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′-N-ethylcarboxamido adenosine (CGS-21680), N-ethylcarboxamido-adenosine (NECA), the 5′(N-cyclopropyl)-carboxamidoadenosine, DPMA (PD 129.944) and metrifudil.

In one embodiment the composition comprises an active ingredient that addresses oily skin. These actives can be sebo-regulating or antiseborrhoeic agents capable of regulating the activity of sebaceous glands. These include: retinoic acid, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate;—derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name SEBUSTOP by Solabia—extracts of flaxseed such as that sold under the name LINUMINE by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name REGU SEB by Pentapharm—mixtures of extracts of willowherb, of Terminalia chebula, nasturtium and of bioavailable zinc (microalgae), such as that sold under the name SEBORILYS GREEN TECH;—extracts of Pygeum afrianum such as that sold under the name PYGEUM AFRIANUM STEROLIC LIPID EXTRACT by Euromed—extracts of Serenoa serrulata such as those sold under the name VIAPURE SABAL by Actives International, and those sold by the company Euromed—of extracts of plantain blends, Berberis aquifolium and sodium salicylate 20 such as that sold under the name SEBOCLEAR RAHN—extract of clove as that sold under the name CLOVE EXTRACT POWDER by Maruzen—argan oil such as that sold under the name LIPOFRUCTYL LABORATORIES SEROBIOLOGIQUES; 25—lactic protein filtrates, such as that sold under the name NORMASEB by Sederma—the seaweed laminaria extracts, such as that sold under the name LAMINARGHANE by Biotechmarine—oligosaccharides seaweed Laminaria digitata, such as that sold under the name PHYCOSACCHARIDE 30 AC by the company Codif—extracts of sugar cane such as that sold under the name POLICOSANOL by the company Sabinsa, the sulfonated shale oil, such as that sold under the name ICHTYOL PALE by Ichthyol—extracts of ‘meadowsweet (Spiraea ulmaria) such as that sold under the name CYTOBIOL ULMAIRE by societeLibiol—sebacic acid, especially sold in the form of a sodium polyacrylate gel under the name SEBOSOFT by Sederma—glucomannans extracted from konjac tuber and modified with alkylsulfonate chains such as that sold under the name BIOPOL BETA by Arch Chemical—extracts of Sophora angustifolia, such as those sold under the name SOPHORA POWDER or Sophora extract by Bioland—extracts of cinchona bark succirubra such as that sold under the name RED BARK HS by Alban Muller—extracts of Quillaja saponaria such as that sold under the name 15 PANAMA WOOD HS by Alban Muller—glycine grafted onto an undecylenic chain, such as that sold under the name LIPACIDE UG OR by SEPPIC—the mixture of oleanolic acid and nordihydroguaiaretic acid, such as that sold under the form of a gel under the name AC.NET by Sederma; 20—phthalimidoperoxyhexanoic acid—citrate tri (C12-C13) sold under the name COSMACOL ECI by Sasol; trialkyl citrate (C14-C15) sold under the name COSMACOL ECL by Sasol—10-hydroxydecanoic acid, including mixtures acid-hydroxydecanoic October 25, sebacic acid and 1,10-decandiol such as that sold under the name ACNACIDOL BG by Vincience and mixtures thereof.

Other skin active agents useful herein include those selected from N-acetyl D-glucosamine, panthenol (e.g., DL panthenol available from Alps Pharmaceutical Inc.), tocopheryl nicotinate, benzoyl peroxide, 3-hydroxy benzoic acid, flavonoids (e.g., flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate: DL-α-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

In a preferred embodiment, the cosmetic composition includes one or more skin active agents selected from free ceramides, e.g., Ceramide EOP, Ceramide AS, Ceramide AP, Ceramide NS, Ceramide NP, Ceramide NH, Ceramide AH, Ceramide EOH, Ceramide EOS, CeramideAdS, Ceramide NdS, and Ceramide EOdS, protein bound ceramides, Phytosphingosine, Sphingosine, ceramide precursors, fatty acids, fatty alcohols, cholesterol, cholesterol sulfate, or combinations thereof.

In various embodiments, the total amount of skin active agents in the cosmetic composition of the disclosure, may be in an amount greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.1 to about 10 wt. %, about 0.1 to about 5 wt. %, about 0.1 to about 2.5 wt. %, about 0.1 to about 1 wt. %, about 0.1 to about 0.5 wt. %; about 1 to about 10 wt. %, about 1 to about 8 wt. %, about 1 to about 6 wt. %, about 1 to about 5 wt. %, about 1 to about 4 wt. %, about 1 to about 3 wt. %; about 2 to about 10 wt. %, about 2 to about 8 wt. %, about 2 to about 6 wt. %, about 2 to about 5 wt. %, about 2 to about 4 wt. %; about 3 to about 10 wt. %, about 3 to about 8 wt. %, about 3 to about 6 wt. %, about 3 to about 5 wt. %; about 4 to about 10 wt. %, about 4 to about 8 wt. %, or about 4 to about 6 wt. %, based on the total weight of the cosmetic composition.

In an embodiment, the compositions form part of a device comprising a cosmetic composition according to the instant disclosure. In an embodiment, the compositions are received in a kit, which is a device, for example, a device that dispenses the cosmetic compositions. In an embodiment, the device with rollerball cartridges comprises composition that is compatible with the said rollerball cartridges, the composition comprising:
a viscosity below 0.34 Pa·s at 25° C., and shear rate of 200 s-1 at 25° C.; and
wherein the cosmetic composition include:
one or more pearlescent pigments; and
a thickening system including at least one synthetic polymer and at least one thickening polysaccharide;
wherein the composition is free of emulsifiers;
wherein the one or more pearlescent pigments and the thickening system form a stable suspension; and
wherein the thickening system is present in an amount sufficient to cause the composition to be retained within a rollerball cartridge assembly without substantial leakage.

In one embodiment, a device including a cartridge that is configured to dispense the cosmetic compositions and retain the cosmetic composition without leaking. The rollerball cartridge may be a cylindrical tube component with glass ball attached to the top. A paired device will push the bottom piston to help dispensing accurate amount. As you roll the ball against skin, the composition will be dispensed.

Example of suitable systems can be cartridges, and dispensing apparatus/devices that are disclosed in U.S. Pat. Nos. 10,842,241; 10,939,740; 9,144,690; which are all incorporated herein by reference in their entirety.

### EXAMPLES

As various changes could be made in the above-described methods without departing from the scope of the invention, it is intended that all matter contained in the above description and in the examples given below, shall be interpreted as illustrative and not in a limiting sense.

The following Examples are intended to be non-restrictive and explanatory only. The ingredient amounts in the compositions/formulas described below are expressed in % by weight, based on the total weight of the composition.

### Example 1

### (Inventive Compositions)

TABLE 1
Inventive Examples

| | INCI US | A | B | C | D |
|---|---|---|---|---|---|
| Pearlescent | Titanium Dioxide (And) Mica | 0.45 | 0.45 | 0.45 | 0.45 |
| pigment | | 0.15 | 0.15 | 0.15 | 0.15 |
| Synthetic | Acrylates/C10-30 Alkyl Acrylate | 0.40 | 0.30 | 0.20 | 0.30 |
| Polymer | Crosspolymer | | | | |
| Synthetic | Ammonium Polyacryloyldimethyl | 0.30 | 0.30 | 0.30 | 0.30 |
| Polymer | Taurate | | | | |
| Polysaccharide | Sodium Hyaluronate | 0.15 | 0.15 | 0.15 | 0.15 |
| Polysaccharide | Xanthan Gum | 0.24 | 0.24 | 0.24 | 0.24 |
| Solvent | Propylene Glycol; Butylene | ~18 | ~18 | ~18 | ~18 |
| | Glycol; Alcohol Denat.; Glycerin | | | | |
| Chelator | | 0.30 | 0.30 | 0.30 | 0.30 |
| Active | Niacinamide; Capryloyl Salicylic | ~22 | ~22 | ~22 | ~22 |
| | Acid; Tocopherol; | | | | |
| | Hydroxyacetophenone; | | | | |
| | Taraxacum Officinale | | | | |
| | (Dandelion) Rhizome/Root | | | | |
| | Extract; Saccharomyces | | | | |
| | Ferment Filtrate; Tropaeolum | | | | |
| | Majus Flower/Leaf/Stem Extract; | | | | |
| Floral Water | Rosa Damascena Flower Water | | 35.0 | | |
| Preservative | | 0.3 | 0.3 | 0.3 | 0.3 |
| PH Adjuster | | 0.00 | 0.15 | 0.10 | 0.15 |
| | Water | Q.S. | Q.S. | Q.S. | Q.S. |

### Example 2

### (Comparative Compositions)

TABLE 2
Comparative Examples

| | | Example 5 | Example 6 | Example 7 | Example 8 | Example 9 |
|---|---|---|---|---|---|---|
| INCI US | | Comparative | Comparative | Comparative | Comparative | Comparative |
| Titanium Dioxide | Pearlescent | 0.45 | 0.45 | 0.45 | 0.45 | 0.45 |
| (And) Mica | pigment | | | | | |
| Titanium Dioxide | Pearlescent | 0.15 | 0.15 | 0.15 | 0.15 | 0.15 |
| (And) Mica | pigment | | | | | |
| Acrylates/C10-30 | Polymer | 0.10 | 0.20 | 0.20 | 0.20 | |
| Alkyl Acrylate | | | | | | |
| Crosspolymer | | | | | | |
| Ammonium | Polymer | 0.30 | 0.30 | 0.30 | | 0.30 |
| Polyacryloyldimethyl | | | | | | |
| Taurate | | | | | | |
| Sodium Hyaluronate | Polysaccharide | 0.15 | | 0.15 | 0.15 | 0.15 |
| Xanthan Gum | Polysaccharide | 0.24 | 0.24 | | 0.24 | 0.24 |
| Water | | 59.33 | 59.29 | 59.38 | 59.44 | 59.34 |
| Results | | Pearl | Very Slight | A Lot Of | Pearl | Pearl |
| | | Settlement | Pearl | Pearl | Settlement | Settlement |
| | | | Settlement | Settlement | without | without |
| | | | without | without | AMPS | Acrylates/C10- |
| | | | sodium | xantham gum | | 30 Alkyl |
| | | | hyluronate | | | Acrylate |
| | | | | | | Crosspolymer |

### Example 3

A comparative study for different thickeners in the system was performed to show the unexpected results from the combination of the different thickeners. The data demonstrated that the thickeners were needed within a certain range and within a certain ratio. The concentration of the pearlescent pigments was kept the same.

In each of the Comparative Examples, one of the mandatory ingredients was removed to show that each ingredient claimed was necessary to obtain compositions that would not show settlement of the pearlescent pigments and would keep the viscosity needed.

The amount of Acrylates/C10-30 Alkyl Acrylate Crosspolymer in Comparative Example 1 showed that if the concentration is below 0.2%, the composition was not stable, and the settlement of the pearl agents were observed.

In Comparative Example 6, it was observed that without the presence of sodium hyaluronate the composition was not stable and the pearl agents settled slightly.

In Comparative Example 7, it was observed that without the presence of the xanthan gum, the composition was not stable and a good quantity of pearl agents settled.

In Comparative Example 8, it was observed that without the presence of ammonium polyacryloylmethyl taurate, the composition was not stable and settlement of the pearl agents were observed.

In Comparative Example 9, it was observed that without the presence of acrylates/C10-30 alkyl acrylate crosspolymer, the composition was not stable and settlement of the pearl agents were observed.

### Example 4

### (Viscosity Measurement)

The viscosity was measured at 25° C., using a Rheomat R180 viscometer equipped with a M2 or M3 spindle, the measurement being performed after 10 minutes of rotation of the spindle in the composition (after which time stabilization of the viscosity and of the spin speed of the spindle are observed), at a shear rate of 200 rpm. The M2 spindle is smaller than the M3 spindle. Accordingly, a viscosity of 60 UD (Deviation Units) measured with a M2 spindle is lower than a viscosity of 27 UD measure with a M3 spindle.

The viscosity was also measure with a rheometer in case of Inventive Example B and Commercial products. The results are shown below and expressed in Pa·s and were measure at 200 1/s.

TABLE 3
Viscosity Measurements

| | Inventive | Commercial | Commercial | Commercial | Commercial |
|---|---|---|---|---|---|
| | Example B | Product 1 | Product 2 | Product 3 | Product 4 |
| Pearl | 0.60% | 0.50% | 0.50% | 0.70% | 0.30% |
| ACRYLATES/C10-30 | 0.40% | 0.20% | 0.20% | 0.15% | 0.25% |
| ALKYL ACRYLATE | | | | | |
| CROSSPOLYMER | | | | | |
| Viscosity (UD) | 60 UD | 27 UD | 27 UD | 25 UD | 42 UD |
| | (M2) +/− 10 | (M3) +/− 5 | (M3) to 37 | (M3) +/− 5 | (M3) +/− 7 |
| | | | UD (M3) | | |
| Viscosity(Pa · s at | 0.25 | 0.40 | N/A* | N/A* | N/A* |
| 200 1/s) | | | | | |
| Consistency | Flowable | Cream | Cream | Cream | Cream |
| | (Water like) | | | | |

*= N/A = Not Available

The Commercial products above contained a structure similar to the Inventive Example B. Indeed, the Inventive Example B and the Commercial Products contained pearlescent pigments as well as Acrylates/C10-30 Alkyl Acrylate Crosspolymer. It was observed that the Inventive Example B and the Commercial Products were all stable and the pearlescent pigments were in suspension. However, after measurement of the viscosities, it was surprisingly observed that the viscosity of the Inventive Example B was much lower than the viscosities of the Commercial Products even though they all had the same structure. The viscosity of Inventive Example B was measured to be 60 UD with a M2 spindle. The viscosities of the Commercial products 1, 2, 3 and 4 were measured to be 27 UD, 27 UD to 37 UD, 25 UD and 42 UD, respectively, with a M3 spindle. As described above, since the spindle M2 is smaller that the spindle M3, even though the viscosity numbers measured with spindle M2 can be higher than the viscosity numbers measured with spindle M3, the viscosity is lower.

The foregoing description illustrates and describes the invention. The disclosure shows and describes only the preferred embodiments but it should be understood that the invention is capable to use in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein.

### Definitions

As used herein, the term “flowable” means maintaining a unique rheology compatible with rollerball cartridges. The viscosity of the compositions according to the instant disclosure can be measured at 25 C, using a ProRheo R180 viscometer equipped with a spindle M2 or M3 rotating at 200 rpm. The viscosity of the composition according to the instant disclosure can be measured and have a range from about 0.15 Pa·s to about 0.35 Pa·s at 25° C., and shear rate of 200 1/s at 25° C. Such defined viscosity is considered “flowable” and is compatible with rollerball cartridges without leaking.

The term “HLB” refers to Hydrophile-Lipophile Balance, which is a measure of the degree to which an emulsifier is hydrophilic or lipophilic, determined by calculating values for the different regions of the molecule, as described by Griffin, William C., “Classification of Surface-Active Agents by ‘HLB’”, JOURNAL OF THE SOCIETY OF COSMETIC CHEMISTS, 1 (5): 311-26 (1949) and Griffin, William C., “Calculation of HLB Values of Non-Ionic Surfactants,” JOURNAL OF THE SOCIETY OF COSMETIC CHEMISTS, 5 (4): 249-56 (1954), which are incorporated herein by reference in their entirety.

“Sugar ester” as used herein means “sugar alcohol fatty acid ester” or “sugar acid fatty alcohol ester” and “sugar ether” as used herein means “sugar alcohol fatty alcohol ether”.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements chosen from A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

An “alkyl radical” is a linear or branched saturated hydrocarbon-based group, particularly C₁-C₈, more particularly C₁-C₆, preferably C₁-C₄ such as methyl, ethyl, isopropyl and tert-butyl;

An “alkoxy radical” is a alkyl-oxy wherein alkyl is as described herein before;

An “alkenyl radical” is a linear or branched unsaturated hydrocarbon-based group, particularly C₂-C₈, more particularly C₂-C₆, preferably C₂-C₄ such as ethylenyl, propylenyl;

An “alkylene radical” is a linear or branched divalent saturated C₁-C₈, in particular C₁-C₆, preferably C₁-C₄ hydrocarbon-based group such as methylene, ethylene or propylene.

Some of the various categories of components identified for the cosmetic compositions may overlap. In such cases where overlap may exist and the composition/product includes two overlapping components (or more than two overlapping components), an overlapping component does not represent more than one component. As an example, a fatty acid may be considered both a “non-triglyceride and non-aromatic fatty emollient” and a “surfactant/emulsifier.” If a particular composition/product includes both a non-triglyceride and non-aromatic fatty emollient component and an surfactant/emulsifier component, a single type of fatty acid can serve as only a non-triglyceride and non-aromatic fatty emollient or a surfactant/emulsifier (a single fatty acid does not serve as both the non-triglyceride and non-aromatic fatty emollient component and the surfactant/emulsifier component).

All percentages, parts and ratios herein are based upon the total weight of the compositions of the present invention, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically points 1, 2, 3, 4 and 5, as well as sub-ranges such as 2-5, 3-5, 2-3, 2-4, 1-4, etc.; and points of 1, 2, 3, 4, and 5 includes ranges and sub-ranges of 1-5, 2-5, 3-5, 2-3, 2-4, 1-4, etc.

Other than in the operating examples, or where otherwise indicated, all numbers expressing quantities of ingredients and/or reaction conditions may be modified with the term “about,” whether or not expressly stated.

Additionally, all numbers are intended to represent exact values as additional embodiments, whether or not modified by the term “about.” For example, “an amount of about 1%” can be modified to refer to exactly 1%. As a further example, “an amount of 1%” can be modified to refer to “about 1%.” Unless otherwise indicated, the term “about” is understood to encompass a range of +/−10% from the stated number. However, in some embodiments, the term may be defined to encompass narrower ranges, for example, +/−1%, 2%, 3%, 4%, 5%, 6%, 7%, 8%, 9%, and 10% from the stated number.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
one or more pearlescent pigments; and
a thickening system including at least one synthetic polymer and at least one thickening polysaccharide;
wherein the composition is free of emulsifiers;
wherein the one or more pearlescent pigments and the thickening system form a stable suspension; and
wherein the thickening system is present in an amount sufficient to cause the cosmetic composition to be retained within a rollerball cartridge assembly without substantial leakage.

2. The composition of claim 1, wherein the one or more pearlescent pigments is selected from the group consisting of titanium mica coated with an iron oxide, mica coated with bismuth oxychloride, titanium mica coated with chromium oxide, nacreous pigments based on bismuth oxychloride, mica coated with titanium dioxide and iron oxides, mica coated with titanium dioxide, and mica coated with titanium dioxide and tin oxide, and combinations thereof.

3. The composition of claim 2, wherein the one or more pearlescent pigments are present from about 0.1 wt. % to about 5 wt. % based on the total weight of the cosmetic composition.

4. The composition of claim 2, wherein the one or more pearlescent pigments is present from about 0.15 to about 4.8 wt. % based on the total weight of the cosmetic composition.

5. The composition of claim 1, wherein the thickening system comprises at least one synthetic polymer selected from the group consisting of acrylates/C10-30 alkyl acrylate crosspolymer, hydroxyethyl acrylate/sodium acryloyldimethyl taurate copolymer (may be 89% active), polyacrylate crosspolymer-6, acrylamide/sodium acryloyldimethyltaurate copolymer, ammonium acryloyldimethyltaurate/steareth-25 methacrylate crosspolymer, acrylates/beheneth-25 methacrylate copolymer, ammonium polyacryloyldimethyl taurate, sodium polyacrylate, carbomer, and combinations thereof.

6. The composition of claim 5, wherein the at least one synthetic polymer is present from about 0.10 to about 1.0 wt. % based on the total weight of the cosmetic composition.

7. The composition of claim 6, wherein the at least one synthetic polymer is present from about 0.15 to about 0.9 wt. % based on the total weight of the cosmetic composition.

8. The composition of claim 1, wherein the thickening system comprises at least one thickening polysaccharide selected from the group consisting of gums, cellulose derivatives, and combinations thereof.

9. The composition of claim 8, wherein the thickening system comprises at least one thickening polysaccharide selected from the group consisting of sclerotium gum, xanthan gum, cellulose gum, locust bean gum, carrageenan, modified celluloses (ethyl, isopropyl, methyl), acacia gum, sodium hyaluronate, and combinations thereof.

10. The composition of claim 9, wherein the at least one thickening polysaccharide is present from about 0.1 to about 0.4 wt. % based on the total weight of the cosmetic composition.

11. The composition of claim 1, wherein the cosmetic composition is a stable suspension as determined by centrifugation at 900 rpm, at 25° C., for one hour.

12. The composition of claim 1, wherein the thickening system amount comprises from about 0.10 to about 1.0 wt. % based on the total weight of the cosmetic composition of at least one synthetic polymer; and from about 0.1 to about 0.4 wt. % based on the total weight of the cosmetic composition of at least one thickening polysaccharide; the said amount is sufficient to achieve a viscosity of about 0.15 to about 0.34 Pa·s, measured at 25° C. at a shear rate of 200 1/s.

13. The composition of claim 1, wherein the viscosity of about 0.15 to about 0.34 Pa·s, measured at 25° C. at a shear rate of 200 1/s is sufficient to cause the cosmetic composition to be retained within a rollerball cartridge assembly without substantial leakage.

14. The composition of claim 1, wherein the cosmetic composition is retained within a rollerball cartridge assembly without substantial leakage when the rollerball cartridge is in an inverted, idle position for one month at 45° C.

15. The composition of claim 1, wherein the cosmetic composition comprises a gel composed of a gelling agent amount of the thickening system, a solvent, and the one or more pearlescent pigments.

16. The composition of claim 14, wherein the solvent comprises at least one of the solvent chosen from glycerin, C2-C6 mono-alcohols, polyols (polyhydric alcohols), glycols, and a mixture thereof.

17. The composition of claim 1, further comprising one or more skin active agents.

18. The composition of claim 1 having a viscosity of about 0.15 to about 0.35 Pa·s at 25° C., and shear rate of 200 1/s at 25° C.

19. A device with rollerball cartridges comprising a cosmetic composition that is compatible with the said rollerball cartridges, the cosmetic composition comprising:
a viscosity below 0.34 Pa·s at 25° C., and shear rate of 200 1/s at 25° C.; and
wherein the cosmetic composition includes:
one or more pearlescent pigments; and
a thickening system including at least one synthetic polymer and at least one thickening polysaccharide;
wherein the cosmetic composition is free of emulsifiers;
wherein the one or more pearlescent pigments and the thickening system form a stable suspension; and
wherein the thickening system is present in an amount sufficient to cause the cosmetic composition to be retained within a rollerball cartridge assembly without substantial leakage.
